(19) United States
(12) Patent Application Publication
(10) Pub. No.: US 2017/0216614 A1
(43) Pub. Date: Aug. 3, 2017

(54) DEVICE FOR BIOLOGICALLY DECONTAMINATING PERCUTANEOUS ACCESS POINTS AND METHOD THEREFOR

(71) Applicant: Leibniz Institut für Plasmaforschung und Technologie Greifswald e.V., Greifswald (DE)

(72) Inventors: Klaus-dieter WELTMANN, Binz (DE); Manfred STIEBER, Greifswald (DE); Stephan KRAFCZYK, Greifswald (DE); Thomas VON WOEDTKE, Gerdeswalde (DE)

(21) Appl. No.: 15/313,558

(22) PCT Filed: May 28, 2015
(86) PCT No.: PCT/EP2015/061900

§ 371 Date: Nov. 23, 2016

(30) Foreign Application Priority Data
May 28, 2014 (DE) 10 2014 107 554.4

(51) Int. Cl. A61N 1/44

(52) U.S. Cl. CPC A61N 1/44

(57) ABSTRACT

Disclosed is a device (1) for biologically decontaminating percutaneous accesses or the stomata of patients, by means of a plasma generator (4) for generating a decontaminating plasma adjacently to a treatment surface (3) of the plasma generator (4). In order to at least partially surround a percutaneous access or stomata, the treatment surface (3) is curved to be adaptable and can be placed against the percutaneous access or stoma.

The invention relates to a device for biological decontamination of percutaneous access points or stomata in patients with a plasma generator for generating a decontaminating plasma adjacent to a treatment surface of the plasma generator.

In medical practice, a wide variety of percutaneous (percutaneous=through the skin) accesses to organs and different regions within the human body are needed for various applications. For this purpose, e.g. catheters of all types (such as bladder catheters, urethral catheters, port catheters, tube catheters, heart catheters, dialysis catheters, etc.) and various types of external drainages (such as a redon drainage, capillary drainage, Shirley drainage, T-drainage, pancreas drainage, etc.) are used that are made of a special tubing material, and serve to drain bodily fluids such as blood, lymph, wound secretions and pus that can arise in particular after surgeries, from the interior of the body to the exterior.

Moreover, surgically placed artificial openings in the surface of the body (so-called stomata) are used in numerous surgeries. Stomata are for example necessary to drain the content of hollow organs to the outside (enterostoma, colostoma, ileostoma, urostoma, tracheostoma, gastrostoma) such as in the case of an artificial intestinal displacement (enterostoma) or artificial urine diversion (urostoma), etc., in the implantation of endo-exo prostheses in which a bone-anchored “endoshaft” is guided through the skin to the outside after the amputation of extremities for mounting an external “exoprosthesis”, or to place percutaneous control cables (drivelines) that are for example used to connect an implanted ventricular support system (VAD—ventricular assist device: a type of artificial heart) to the external control device.

A problem with the placement of such accesses and stomata is the risk of infection of the affected skin area. The risk of infection in this region is particularly high since the skin as the largest bodily organ provides germs in particular with a large area of attack, and skin germs can penetrate through the catheter or drainage entry point into the body where they can cause complications such as the formation of an abscess or life-threatening sepsis (blood poisoning). Therefore, the avoidance or reduction of the risk of infection in placement has a significant role in the follow-up treatment of percutaneous accesses and stomata of all kinds.

For this purpose, the skin areas surrounding a percutaneous access or stomata are covered with a dry, sterile bandage, inspected regularly, cleaned, treated anti-septically and, when applicable, prophylactically with antibiotics. Furthermore, for example when the skin is infected with multi-resistant pathogens, special protective measures are necessary such as a decolonization of the skin.

The residual risk of infection for percutaneous accesses and stomata is high, and a further reduction of the risk is therefore desirable. The use of disinfectants is associated with the problem of potential allergic reactions. Furthermore, disinfection measures are generally time and material-intensive. This applies in particular to prophylactic measures against infection from multi-resistant pathogens. In addition to disinfection measures, measures that promote healing are also necessary to promote wound healing.

It is well known that plasma has a disinfecting effect.

WO 2011/023478 A1 discloses a device for the extensive treatment of regions of human or animal skin or mucosal surfaces, respectively, with cold atmospheric pressure plasma. For this purpose, a sleeve with an electrode array installed in the sleeve is wound around a body part. The region to be treated is completely covered with the sleeve.

A device is known from WO 2013/167693 A1 for the plasma treatment of human, animal or plant surfaces, in particular of the skin or areas of mucosa. The device has an electrode array in which the high voltage electrode consists of a thin wire and is sheathed with an insulation layer. The grounded electrode consists of a flat, gas-permeable textile surface structure that can be flexibly placed on any curved surfaces.

Proceeding from the above, it is the object of the present invention to provide a device for the biological decontamination of percutaneous accesses or stomata in patients.

The object is achieved with a device having the features of claim 1.

Advantageous embodiments are described in the subclaims.

The use of decontaminating plasma for the biological decontamination of percutaneous accesses or stomata is therefore proposed to render the prophylaxis of infection easier and more effective, and moreover to promote wound healing. This is accomplished in that a device for biologically decontaminating percutaneous accesses or stomata in patients has a plasma generator with a treatment surface which provides a decontaminating plasma, wherein this treatment surface is adaptably curved to at least partially surround a percutaneous access or stoma, and can be placed on the percutaneous access or stoma.

The risk of infection through percutaneous accesses or stomata can be reliably and easily reduced with the treatment surface of a plasma generator that at least partially surrounds a percutaneous access or stomata, and by placing this adaptively curved treatment surface on the percutaneous access or stoma. For this purpose, the plasma generator can preferably be designed to generate a cold atmospheric pressure plasma at the treatment surface. For example, plasma jet arrays, a DBD array (DBD=dielectric barrier discharge) or corona arrays are in principle suitable plasma sources.

A significant minimization of the infection risk in the region of percutaneous accesses and stomata can be achieved by the device. Furthermore, allergic reactions are avoided that otherwise frequently occur when disinfecting percutaneous accesses and stomata with disinfectants. Furthermore, infections from multi-resistant pathogens can be effectively avoided with the expenditure of comparatively less time and materials. The device allows considerable time and materials savings in the use of percutaneous accesses or stomata in comparison to conventional infection prophylaxis. Furthermore, the device brings about a promotion of wound healing since the plasma used for decontamination is both decontaminating and promotes healing.

In a preferred embodiment, a tubular percutaneous access or tubular stoma has a treatment surface integrally connected thereto on the outside of a flexible tube used to introduce or drain fluids. Accordingly, during the time of using the percutaneous access or stoma, the tissue region adjacent to the treatment surface can be treated with plasma continuously or repeatedly without additional installation effort. The device is therefore a percutaneous access or stoma with a tubular structure that has a plasma treatment surface which is curved to be adaptable to the tubular structure, and at least partially surrounds this tubular structure.

However, an embodiment is also conceivable in which the treatment surface is cylindrical, and the interior of the cylindrical treatment surface is provided to accommodate a tube for a percutaneous access or stoma. Before using the percutaneous access or stoma, the treatment surface of the plasma generator is accordingly shoved onto the tube in order to be subsequently activated for decontamination continuously or repeatedly at intervals during use.

It is also conceivable for the device for the biological decontamination of percutaneous accesses or stomata to be designed to be used temporarily for biological decontamination. In this embodiment, the device preferably has a holder with a handle and a recess with a U-shaped curve. The treatment surface of the plasma generator lies against the inner wall of the recess with the U-shaped curve. In this manner, the treatment surface with a U-shaped curve can be placed adjacent to the percutaneous access or stoma in the region of the tissue surface such that the percutaneous access or stoma is inserted in the recess with a U-shaped curve and borders the treatment surface. Then, after the plasma generator is activated, a decontaminating plasma, such as preferably cold atmospheric gas plasma is generated by the treatment surface, and the percutaneous access or stoma and adjacent skin region are thereby decontaminated. This has a simultaneously wound promoting effect on the tissue that is adjacent to the percutaneous access and stoma.

Another device suitable for temporary decontamination is preferably characterized in that the device has pliers with two pliers jaws that are mounted movable relative to each other. A treatment surface is arranged on each of the facing inner surfaces of the pliers jaws. In this manner, the treatment surface can surround the tubular structure of a percutaneous access or stoma and be positioned adjacent thereto. With the assistance of the pliers, the treatment surface can be adapted to different diameters.

Integrating a plasma treatment surface of a plasma generator with the pliers jaws allows a decontamination device to be easily handled so that percutaneous accesses and stomata in particular can be biologically decontaminated very easily and reliably.

With all of the cited embodiments of the device, it is particularly advantageous when the treatment surface has a flexible holder with at least one high-voltage electrode enclosed by the flexible holder, and at least one ground electrode on the outer surface of the flexible holder. The flexible holder is formed from a suitable insulating material such as a dielectric, and is arranged between the high-voltage electrode and the ground electrode. The ground electrode is located on an outer surface of the flexible holder which is provided for arrangement adjacent to the percutaneous access or stoma. Accordingly, when a high voltage is applied to the high-voltage electrode, a cold atmospheric gas plasma is generated by a plasma generator at the outer surface of the flexible holder bearing the ground electrode. This plasma can then act on the adjacent percutaneous access, an adjacent stoma, and the tissue regions of a patient adjacent thereto. The patient can be a human or animal.

The ground electrode is preferably a wire winding wound around the flexible holder. The high-voltage electrode is covered on both sides by a flexible holder, and is therefore arranged in the gap of a flexible holder. Given a tubular treatment surface, this causes both the cylindrical inner surface as well as the outer surface to have a ground electrode. This reduces the danger from electrical high-voltage.

An outer surface of the flexible holder provided with a ground electrode can therefore for example be placed in a curved, U-shaped recess of a holder, or on a jaw of pliers that is thereby electrically grounded.

At least one ground electrode should, however, be arranged on the surface of the treatment surface provided for placement on the percutaneous access or stoma.

It is particularly advantageous to design the high-voltage electrode as a flexible wire electrode. It can however also be formed from a mesh element, for example. The measuring electrode can also be designed as an electrically conductive mesh element.

It is particularly advantageous when the flexible holder is formed from a dielectric plastic material, in particular polyimide. Such a dielectric plastic material is particularly suitable for generating plasma by means of high voltage. The polyimide ‘Kapton® CR’ by Du Pont is distinguished by a particularly high, stable electric strength and plasma resistance.

The invention is explained in greater detail below using exemplary embodiments with accompanying drawings. The figures show the following:

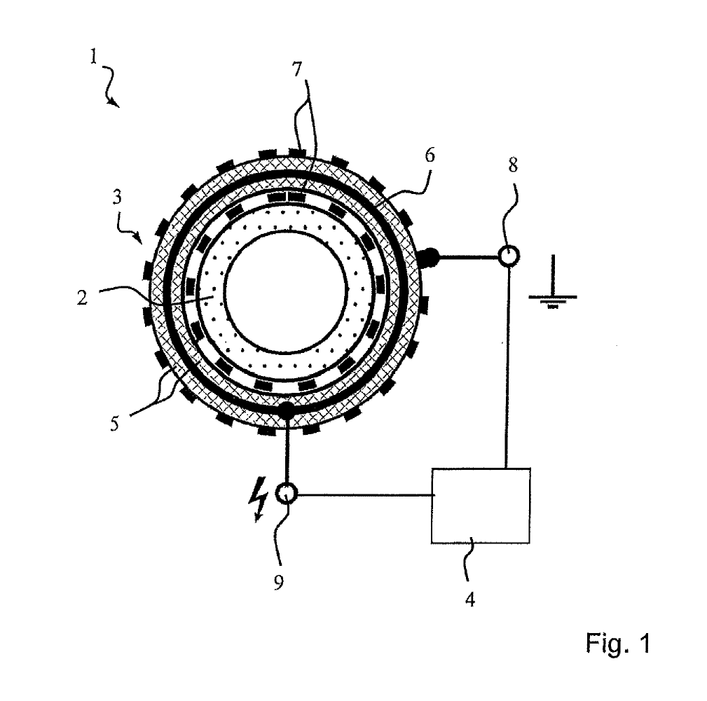

FIG. 1 Cross-sectional view of a first embodiment of a device for biologically decontaminating percutaneous accesses or stomata with a tubular structure that is surrounded by a plasma treatment surface;

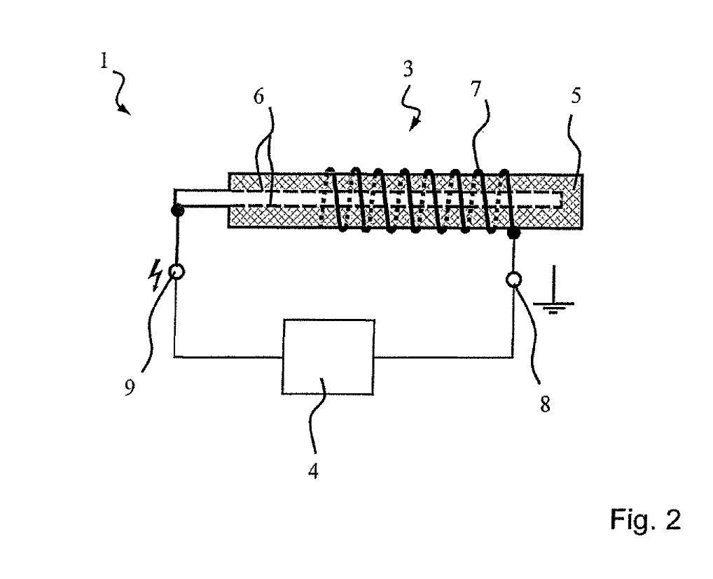

FIG. 2 Lateral section of a device for biological decontamination that is flexibly adaptable to a percutaneous access or a stoma;

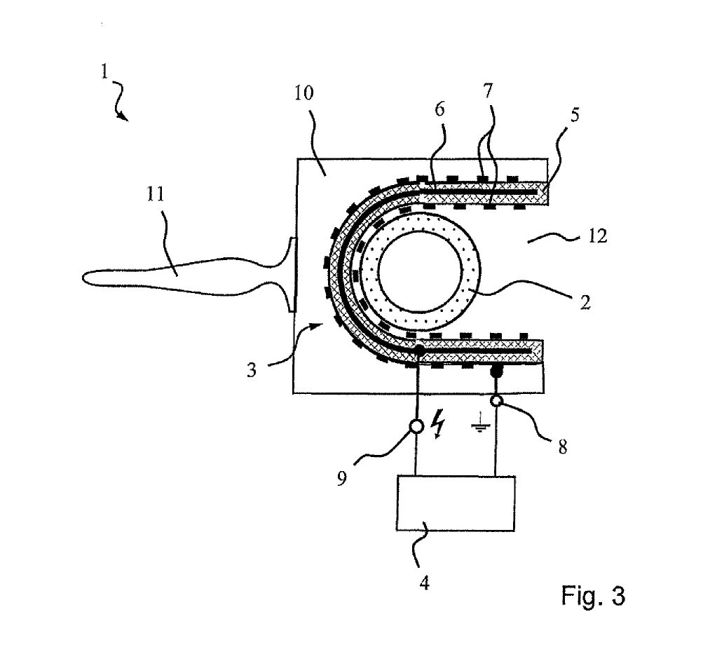

FIG. 3 Plan view of an embodiment of a manually-guided device for biologically decontaminating percutaneous accesses or stomata;

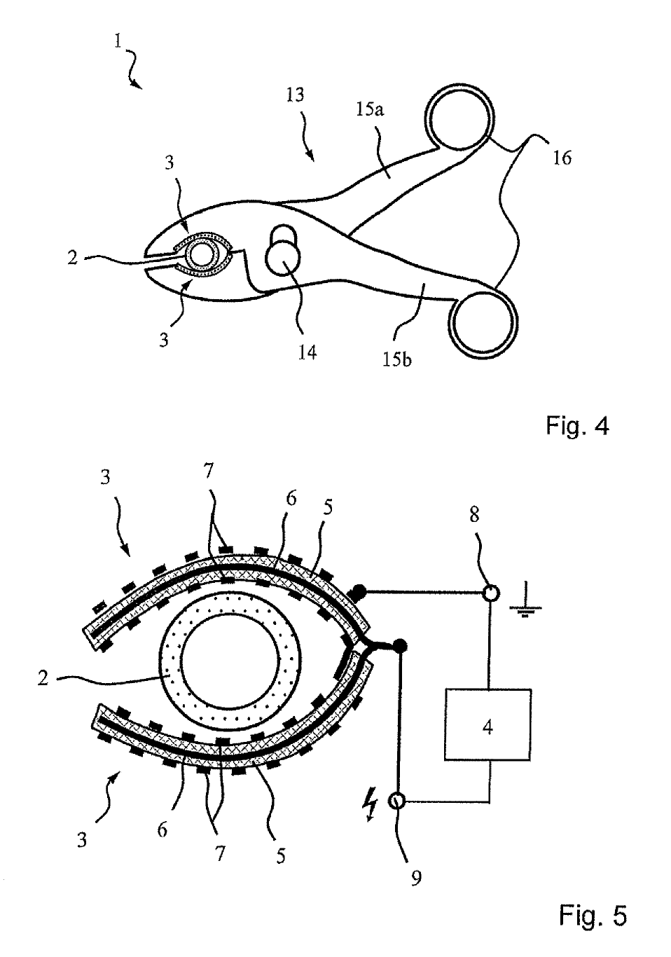

FIG. 4 Sketch of a device designed as pliers for biologically decontaminating percutaneous accesses or stomata;

FIG. 5 Sketch of the treatment surface integratable in the jaws of the pliers from FIG. 4.

FIG. 1 shows a device 1 for biologically decontaminating percutaneous accesses or stomata. Such a percutaneous access or stoma has a tubular structure 2, on the outer perimeter of which a treatment surface of a plasma generator is arranged. The plasma generator itself is a generator that generates a high-voltage as indicated by the high-voltage arrow.

The treatment surface 3 has an insulating jacket 5 surrounding the tubular structure 2 in which a high-voltage electrode 6 is embedded. A ground electrode 7 is arranged on each of the two outer sides of the insulating jacket 5. This can for example be designed as a wire winding wound around the insulating jacket 5. It is however also conceivable for a fabric or mesh consisting of an electrically conductive material, in particular a metal mesh, to be placed on the cylindrical inner surface and/or the cylindrical outer surface of the insulating jacket 5 and connected in each case to ground potential GND.

To this end, a ground connection 8 that is connected in an electrically conductive manner to the ground electrode 7, as well as a high-voltage connection 9 that is connected in an electrically conductive manner to the high-voltage electrode, run to the outside of the device 1 in order to connect the high-voltage generator 4 to the treatment surface 3.

By applying a high-voltage either as a direct voltage, pulsed direct voltage or alternating voltage to the high-voltage electrode 6, a cold atmospheric gas plasma is generated in a known manner at the ground electrode surfaces. This plasma forms in the air gaps of the ground electrodes 7 and contacts the surface of the tubular structure 2. The plasma flow generated in this matter then reaches the adjacent tissue surfaces. In this manner, biological decontamination occurs of the tubular structure 2 of the percutaneous access or stoma and adjacent tissue. Furthermore, wound healing of the adjacent tissue is improved by the plasma.

FIG. 2 shows a perspective lateral sectional view of a device for generating a dielectric barrier discharge (DBD) with the assistance of a wire arrangement. The high-voltage electrode 6 in this case consists of a wire that is sheathed with a suitable insulating material 5. A metal wire is used as the grounded ground electrode 7 that spirals around the insulating jacket 5 and the high voltage electrode 6 embedded therein. If the high-voltage electrode 6 and ground electrode 7 are connected to a special high-voltage generator, a plasma is generated on the surface of the insulation jacket 5 in the region of the spiral grounded ground electrode 7 upon selecting an appropriate frequency and setting a sufficient voltage level at the high voltage electrode. The advantage of this electrode arrangement is especially that the plasma source can be adapted easily to a wide variety of shapes and sizes of the percutaneous accesses and stomata due to the flexibility of the wire arrangement of the high voltage electrode 5 and the ground electrode 6. Accordingly, the arrangement can for example be wound in a spiral around a tubular structure. It is also conceivable to place the device 1 in the shape of a partial circle or closed circle around the insertion point of a percutaneous access or stoma in a human or animal body in order to continuously or temporarily decontaminate this wound region.

Relevant to the arrangement depicted in FIG. 2 is that the insulation jacket 5, the high voltage electrode 6 and the ground electrode 7 designed as a wire winding together form a flexible arrangement that can be adapted to a curved percutaneous access or stoma.

FIG. 3 shows a cross-sectional view of another embodiment of a device 1 for biologically decontaminating percutaneous accesses in the plan view as a sectional view. The device 1 has a holder 10 and a handle 11 on the holder 10. The holder 10 has a recess 12 with a curved U-shape into which a tubular structure 2 of a percutaneous access or stoma can be inserted. In practice, the device 1 is placed on a percutaneous access or stoma such that the tubular structure 2 is inserted in the U-shaped recess 12. A treatment surface 3 of the plasma generator 4 is arranged on the inside of the curved, U-shaped recess 12. In turn, the treatment surface 3 has an insulation jacket 5 that electrically insulates a high-voltage electrode 6 from the ground electrode 7 and prevents flashover.

The insulation body 5 with its high voltage electrode 6 embedded therein is seated in the recess 12 with the U-shaped curve of the holder 10. A ground electrode 7 that can be connected to the ground potential of a high-voltage generator 4 is arranged on the outer surface of the insulation body 5 that forms the free interior of the curved, U-shaped recess 12 for accommodating the tubular structure 2 of a percutaneous access or stoma.

In the depicted exemplary embodiment, such a ground electrode 7 is also on the opposite side of the insulation jacket 5 adjacent to the handle 10. This ensures that the holder 10 and the handle 11 attached thereto are always connected to the ground potential and grounded. The holder 10 itself can however also consist of an electrically conductive material and form the ground electrode 7.

In the depicted exemplary embodiment, the ground electrode 7 is bound around the insulation jacket 5 as a flexible wire electrode such that the treatment surface 3 can be adapted to the curved, U-shaped recess 12 of the holder 10.

FIG. 4 shows another exemplary embodiment of a device 1 for biologically decontaminating percutaneous accesses or stomata. This device has pliers 13 with two plier handles 15a, 15b that are arranged movable relative to each other about a pivot bearing 14 and each have a jaw 17 on their ends opposite the grip 16. The jaws 17 have a trough-like recess so that they cover a partially-circular or oval, at least partially closed area. In the gap between the jaws 17, a tubular structure 2 of a percutaneous access or stoma can be inserted. The jaws 17 are first opened with the assistance of the handles 156a, 15b, the tubular structure 2 is inserted between the jaws 17, and the pliers 13 are then closed.

It can be seen that a treatment surface 3 is arranged on each facing inner surface of the jaws 17 in the aforementioned manner and is connected to a plasma generator 4 (such as a high-voltage generator). The treatment surface 3 has an insulation jacket 5 in the aforementioned manner that shields a high-voltage electrode 6 against a ground electrode 7. The ground electrode 7 of the treatment surface 3 is arranged on the free inner surface adjacent to the tubular structure 2. Accordingly, for example, cold atmospheric gas plasma is generated on the free inner side of the treatment surfaces 3 in order to thereby biologically decontaminate the tubular structure 2 of a percutaneous access or stoma, as well as tissue adjacent thereto.

FIG. 5 reveals an enlarged view of the treatment surfaces 3 for the device 1 from FIG. 4 in the sectional view. It is clear that the treatment surfaces 3 are curved to adapt to the trough-like recess in the jaws 17. To accomplish this, the treatment surfaces 13 are preferably formed from a flexible holder in the form of the insulation jacket 5. As is the case with the previous embodiments, the insulation jacket 5 accommodates the also adapted, curved high-voltage electrode 6 which can be designed as a curved plate or rod. The ground electrode 7 is arranged at least on the inside of the insulation jacket 5 and is connectable to the ground connection of the high-voltage generator 4. For their part, the high-voltage electrodes 6 of the treatment surfaces 3 facing each other are connected to the high-voltage connection of the high voltage generator 4 (plasma generator).

In the portrayed embodiment, the ground electrodes 7 are in turn designed as wound wire electrodes that surround both sides of the insulation jacket and lie against the top side of the insulation jacket 5, both on the inside as well as on the outside lying against the holder 10.

Other forms of this device are also conceivable. In addition to these depicted wire arrangements in an O-shape, U-shape or pincer-shape, spiral, pin-shape or similar embodiments of the treatment surface are conceivable. Treatment surfaces are also possible that at least partially enclose a tubular structure of a percutaneous access or stoma, or for example can be temporarily placed on the outer perimeter of a percutaneous access or stoma with pliers, wherein plasma generated from a plasma generator is conducted as a stream of gas or fluid through cavities in the treatment surface to the percutaneous access or stoma. With this embodiment, the plasma used for biological decontamination is not generated directly at the treatment surface but rather in the plasma generator. It is conducted from there to the treatment surface.

According to the present invention, a treatment surface can be both a passive treatment surface for distributing plasma generated in a plasma generator, or an active treatment surface for the generation and laminar distribution of plasma.

Biological decontamination of percutaneous accesses or stomata of human or animal patients is carried out with an aforementioned device 1 by means of the steps of
at least partially encompassing the percutaneous access or stoma with a curved treatment surface of a plasma generator;
placing the treatment surface on the percutaneous access or the stoma, and
generating decontaminating plasma at the treatment surface lying against the percutaneous access or stoma.

“Placing” is understood to mean that the treatment surface lies directly against a tubular structure 2 of the percutaneous access or stoma, or it is arranged closely spaced to the tubular structure 2, adjacent with a gap. It is hence unnecessary for the treatment surface 3 to have to touch the percutaneous access or stoma. It must only sit closely such that plasma on the treatment surface 3 contacts the surface of the percutaneous access or the stoma and, if applicable, the adjacent tissue and has a decontaminating effect.

1. A device (1) for biological decontamination of percutaneous accesses or stomata in patients with a plasma generator (4) for generating a decontaminating plasma adjacent to a treatment surface (3) of the plasma generator (4), wherein the treatment surface (3) is curved in an adaptable manner to at least partially surround a percutaneous access or stoma, and can be placed on the percutaneous access or stoma;
wherein the treatment surface (3) has a flexible holder (5) with at least one high-voltage electrode (6) enclosed by the flexible holder (5), and at least one ground electrode (7) on the outer surface of the flexible holder (5) and
wherein the ground electrode (7) is a wire winding wound around the flexible holder (5).

2. The device (1) according to claim 1, characterized in that the device has a holder (10) with a handle (11) and a curved, U-shaped recess (12), and the treatment surface (3) of the plasma generator (4) lies against the inner wall of the curved, U-shaped recess (12).

3. The device (1) according to claim 1, characterized in that the treatment surface (3) is cylindrical, and the interior of the cylindrical treatment surface (3) is provided to accommodate a tube (2) of a percutaneous access.

4. The device (1) according to claim 1, characterized in that the device (1) has pliers (13) with two pliers jaws (17) that are mounted movably relative to each other, and that on each of the mutually facing inner surfaces of the pliers jaws (17) a treatment surface (3) is arranged.

5. (canceled)

6. (canceled)

7. The device according to claim 1, characterized in that the ground electrode (7) is arranged on the outside of the treatment surface (3) provided for placement on the percutaneous access or stoma.

8. The device (1) according to claim 1, characterized in that the high-voltage electrode (6) is a flexible wire electrode.

9. The device (1) according to claim 1, characterized in that the flexible holder (5) is formed from a dielectric plastic material, in particular polyimide.

10. The device (1) according to claim 1, characterized in that the plasma generator (4) with its at least one treatment surface (3) is configured to generate cold atmospheric pressure plasma.

11. A use of a device (1) according to claim 1 for biologically decontaminating percutaneous accesses or stomata in patients.
